(19) United States
(12) Patent Application Publication
(10) Pub. No.: US 2004/0197319 A1
(43) Pub. Date: Oct. 7, 2004

(54) Wound healing composition derived from low platelet concentration plasma

(76) Inventors: Paul Harch, New Orleans, LA (US); James Gandy, West Monroe, LA (US)

Correspondence Address: HESLIN ROTHENBERG FARLEY & MESITI PC, 5 COLUMBIA CIRCLE, ALBANY, NY, 12203, US

(21) Appl. No.: 10/395,707

(22) Filed: Mar. 24, 2003

(51) Int. Cl. A61K 45/00

(52) U.S. Cl. 424/93.72

(57) ABSTRACT

A wound healing composition derived from a low platelet concentration plasma preparation is described. The composition differs from conventional platelet gel preparations in that the centrifugal conditions under which the low platelet concentration plasma is prepared are less stringent than those for the preparation of platelet rich plasma and platelet concentrates. As a result the low platelet concentration plasma preparation from which the wound healing composition of the present invention is prepared has, as its name implies, a concentration of platelets that is lower than the platelet concentration of platelet rich plasma or of whole blood. Methods of making and using the wound healing composition of the present invention are also described.

### FIELD OF INVENTION

The present invention relates to formulations for wound healing and more particularly, to an autologous wound-healing formulation and method for the preparation and use thereof. The formulation of the present invention is prepared using a patient's own plasma which contains not only a lower concentration of platelets as compared to traditional platelet preparations but also contains blood components usually discarded with standard platelet concentration techniques.

### BACKGROUND OF THE INVENTION

Wounds are caused by a disruption in the structural integrity of biological tissue. Such a disruption stimulates the release of thrombin and exposes collagen fibrils, both of which activate blood-borne platelets. Over the past several years, sound clinical and physiological documentation has been obtained indicating that various growth factors that are well known to reside within the alpha granules of platelets have been shown to enhance the body's normal response to injury. Additionally, other cells and cellular components in autologous tissue coagulum, such as plasma proteins (collagen, fibrin, fibronectin, elastin, etc.), white blood cells (WBC), fibroblasts, etc., are known to assist in the wound healing process due to their various chemotactic and mitogenic effects.

In the initial stages of wound healing, fibrinogen is converted to fibrin, forming a fibrin clot, which seals ruptured vessels in the wound and protects the wound. The fibrin clot further forms a matrix or framework for the migration of T-cells, endothelial cells, fibroblasts and epidermal keratinocytes. The activated platelets release granules, which contain various substances such as epidermal growth factor (EGF), vascular endothelial growth factor (VEGF), fibroblast growth factor (FGF), platelet-derived growth factor (PDGF), transforming growth factor beta (TGF-β) etc. all of which facilitate wound healing. Growth factors which have been shown to enhance the body's natural healing process, have been widely characterized. Platelet derived growth factor (PDGF), for example, initiates connective tissue healing including bone regeneration and repair and increases mitogenesis, angiogenesis, and macrophage activation. TGF-β increases chemotaxis and mitogenesis of osteoblast precursors and stimulates osteoblast-mediated deposition of the collagen matrix in wound healing and bone regeneration. Epidermal growth factor (EGF) induces epithelial development and promotes angiogenesis. Vascular endothelial growth factors (VEGF) have potent angiogenic, mitogenic, and vascular permeability-enhancing activities specific for endothelial cells.

Traditionally, autologous platelet gels have been used safely for decades in hospitals and wound care centers, the operating room and physician's office settings to enhance or accelerate wound healing and provide hemostasis at a surgical or wound site. Autologous platelet gels provide supraphysiological quantities of platelet-derived growth factors in a sustained medium at the site of wound healing. The autologous platelets in the gels present huge reservoirs of growth factors that have the potential to greatly accelerate the normal healing process in a natural manner. The use of concentrated growth factors is considered by many to be a new frontier of clinical therapy and has created a paradigm shift affecting all segments of the medical profession.

Traditional platelet gel preparation methods focus on concentrating platelets to increasingly higher amounts to maximize the concentration of endogenous growth factors delivered to the wound site. It is common in most instances and, in fact, viewed as desirable to achieve platelet counts in excess of over three to ten times baseline counts.

In focusing on the concentration of platetets and the growth factors they contain within their alpha granules, the value of the platelet poor plasma fraction which is discarded has been overlooked.

### SUMMARY OF THE INVENTION

The present invention provides a method and formulation for the activation of platelets in a low platelet concentration plasma preparation and application of the platelets at the site of an open wound, a post-operative surgical wound, or intra-operative use at a surgical site. The formulation includes low platelet concentration plasma (LPCP) prepared from a patient's own blood, in addition to one or more platelet-activation agents.

In one aspect, the invention relates to a topical wound healing composition comprising: a low platelet concentration plasma preparation, a platelet activating agent and optionally, CaCl₂ in an amount sufficient to enhance activation of the platelets. The wound healing composition of the present invention may further include a triple copper peptide, glycyl-histidyl-lysine-Cu, in an amount sufficient to enhance healing.

In a related aspect, the invention relates to a method of producing a topical wound healing composition comprising the steps of obtaining whole blood from a patient, obtaining a low platelet concentration plasma preparation from said blood, activating the low platelet concentration plasma preparation and applying the resulting wound healing composition to a wound of the patient.

In yet another aspect, the invention relates to a method for treating a wound of a patient wherein said method comprises obtaining whole blood from the patient; obtaining a low platelet concentration plasma preparation from the collected blood; activating the low platelet concentration plasma preparation with a platelet activating agent to form a wound healing composition; and applying said wound healing composition to the wound of the patient.

### DETAILED DESCRIPTION OF THE INVENTION

All patents, applications, publications, or other references that are listed herein are hereby incorporated by reference.

Methods for the production of platelet rich plasma (PRP) and platelet concentrates (PC) are well known in the art. Generally, for production of platelet-rich plasma, for example, the blood is centrifuged at about 1,200×g for 2.5 minutes and approximately ⅔ of the platelet poor plasma (PPP) is removed and discarded. The platelets are then resuspended in the remaining PPP, thereby creating a very concentrated platelet rich plasma (PRP) solution. In some instances, the platelets are further concentrated by an additional centrifugation step; the platelet poor plasma is removed in its entirety, and the pelleted platelets are resuspended in a small amount of serum or in a non-autologous solution, for example, a buffer or sodium chloride solution. This results in a very concentrated platelet preparation containing little, if any, plasma and its constituent proteins which are critical to wound healing.

Platelet gel formulations known in the art are derived from platelet rich plasma (PRP) or plasma concentrates (PC) and the focus in the platelet gel art has been toward maximization of platelet concentration. PRP comprises approximately 10% of the total blood volume collected and generally has platelet counts of 500,000 to 3,000,000/mm³ depending on the patient's baseline platelet count and the separation method used (See Table 1). For example, assuming a normal whole blood platelet count of 250,000/μl, the PCCS™ platelet concentrate collection system (3i Implant Innovations, Palm Beach Gardens, Fla.) achieves a 10-fold concentration of platelets.

TABLE 1

| | Platelet concentrate or | |
|---|---|---|
| Whole blood | Platelet rich plasma | |
| (platelets/μl) | (platelets/μl) | Method of concentration |
| 2.66 × 10⁵ | 1.41 × 10⁶ | Discontinuous cell |
| | | separation^{a} |
| 2.62 × 10⁵ | 1.42 × 10⁶ | Discontinuous cell |
| | | separation^{b} |
| — | 2.23 × 10⁶ | PCCS PRP^{c} |
| — | 1.14 × 10⁶ | Curasan (“buffy coat”) |
| | | method^{c} |
| 2.38 × 10⁵ (♂) | 1.30 × 10⁶ | Discontinuous plasma |
| | | separation^{d} |
| 2.72 × 10⁵ (♀) | 1.54 × 10⁶ | Discontinuous plasma |
| | | separation^{d} |
| 2.38 × 10⁵ (♂) | 9.44 × 10⁵ | Curasan (“buffy coat”) |
| | | method^{d} |
| 2.72 × 10⁵ (♀) | 1.02 × 10⁶ | Curasan (“buffy coat”) |
| | | method^{d} |

^{a}Weibrich et al., “Growth factor levels in platelet-rich plasma and correlations with donor age, sex, and platelet count,” J. Craniomaxillofac. Surg. Apr; 30(2): 97-102 2002.
^{b}Weibrich et al., “Quantification of thrombocyte growth factors in platelet concentrates produced by discontinuous cell separation,” Growth Factors Jun; 20(2): 93-7 2002.
^{c}Weibrich et al., “Growth factor levels in the platelet-rich plasma produced by 2 different methods: curasan-type PRP kit versus PCCS PRP system,” Int. J. Oral Maxillofac. Implants Mar-Apr; 17(2): 184-90 2002.
^{d}Weibrich et al., “Correlation of platelet concentration in platelet-rich plasma to the extraction method, age, sex, and platelet count of the donor,” Int. J. Oral Maxillofac. Implants Sept-Oct; 16(5): 693-9 2001.

It has now been unexpectedly determined that a wound healing composition prepared from a patient's plasma and comprising less than 100% of the patient's baseline platelet concentration, i.e. a low platelet concentration plasma preparation, is effective in facilitating healing in refractory wounds. Accordingly, the present invention provides a wound healing formulation comprising a plasma preparation having a low platelet concentration relative to the platelet concentrations found in PRP and PC which is prepared under centrifugation conditions that are less stringent than those which result in significant separation of platelets. The platelet preparation of the present invention contains a platelet concentration in the range of about 50,000 to about 500,000/mm³ as opposed to 1,000,000/mm³ and up for PRP and PC.

The term “low platelet concentration plasma (LPCP)” refers to a platelet preparation that is obtained by centrifugation of the collected blood under conditions that are less stringent than those required to obtain platelet-rich plasma (PRP) and platelet concentrates (PC), thereby defining a plasma preparation that is less well resolved in terms of cell separation. Additionally, no specific attempt is made to minimize the amount of platelet poor plasma in the preparation. Rather, for preparation of the low platelet concentration plasma of the present invention, between about 10 to 70% and more preferably between about 30 and 50% of the sample is harvested following centrifugation. As a result, the plasma preparation of the present invention has a significantly greater plasma portion as well as a lower platelet concentration than the platelet concentrates conventionally used for the production of platelet gels in which the platelet poor plasma and proteins contained therein are necessarily removed.

The term “platelet activating agent” refers to a compound which is capable of activating platelets thereby initiating the platelet release reaction. Typical platelet activating agents include, thrombin (either bovine or autologous), trypsin, collagen and thrombin receptor activating peptides etc. For purposes of practicing the methods of the present invention, platelets may be activated by mechanical means as well as by chemical means and the invention is intended to encompass activation by chemical and mechanical means individually or in combination.

Preparation of the low platelet concentration plasma (LPCP) of the present invention requires centrifugation of the collected plasma at a lower g force (about 100-500×g or more preferably about 135-280×g) than for the preparation of PRP and for a shorter time interval (about 10 sec to 5 minutes). Following centrifugation, between about 10% and 70%, and more preferably between about 30% and 50% of the platelet-containing plasma is harvested for use in preparation of the wound healing composition. As a result, platelet concentrations in a LPCP preparation for the wound healing composition of the present invention are in the range of 50,000 to 500,000/μl and more preferably about 80,000 to 200,000/μl depending on the patient's baseline platelet level.

The wound healing formulations of the present invention comprise a low platelet concentration plasma and a platelet activation agent. Not wishing to be bound by theory, the advantage of the lower platelet concentration plasma preparation of the present invention over its high platelet concentrate counterparts is the delivery, when the platelets are activated, of an optimal (physiologic or near-physiologic) amount (as opposed to maximal or supra-physiological amounts) of growth factors necessary for wound healing. Additionally, preparation of a low platelet concentration plasma results in a less rigorous separation of non-cellular plasma components, thereby retaining a greater portion of the essential non-cellular components of the plasma than platelet rich plasma or a platelet concentrate. The low platelet concentration plasma preparation of the present invention therefore, contains other valuable components of the plasma which are generally removed during the preparation of platelet-rich plasma and platelet concentrates. These components are responsible for enhancing the activity of the platelet-derived growth factors and healing of wounds.

In one embodiment, the wound healing preparation of the present invention comprises, in addition to the low platelet concentration plasma, a platelet activator, for example, thrombin in an amount adequate to cause activation of platelets in the low platelet concentration plasma. In one embodiment, the amount of activator for the LPCP is 1,000 units of thrombin. Additionally, an amount of CaCl₂ sufficient to enhance platelet activation, about 0.5 ml to 1 ml of 10% calcium chloride, is added to the wounded healing composition of the present invention.

Synthetic peptides corresponding to at least the first five amino acids of the new N-terminal tail generated after thrombin proteolysis of its receptor are effective to mimic thrombin [Lasne et al., Different abilities of thrombin receptor activating peptide and thrombin to induce platelet calcium rise and full release reaction. Thromb. Haemost., 74(5): 1323-8 (1995)]. Accordingly, in an alternate embodiment, the wound healing composition of the present invention contains as the platelet activating agent an amount of a thrombin receptor activating peptide (TRAP) adequate to cause activation of the platelets in the low platelet concentration plasma.

In yet another embodiment of the invention, activation of platelets in the low platelet concentration plasma is accomplished by mechanical means. For example, a preparation of low platelet concentration plasma is poured into an uncoated glass container such as a glass Petri dish. The platelets in the low platelet concentration plasma are thereby mechanically activated causing the formation of the formulation gel of the present invention, which is applied directly to the wound or to an appropriate bandage or dressing. In a related embodiment, the LPCP is injected into an uncoated glass or a non-glass container through a syringe or cylinder containing uncoated glass beads.

Activation may be accomplished by a combination of chemical and mechanical means. For example, in yet another embodiment, the LPCP is mixed with a concentration of thrombin or thrombin receptor activating peptide (TRAP) adequate to cause activation of the platelets in the LPCP to define a wound-healing formulation. The resulting formulation is then poured into an uncoated glass container to define a formulation gel. Alternatively, the liquid wound-healing formulation can be injected into an uncoated glass or a non-glass container through a syringe or cylinder containing glass beads, which also activate the platelets and cause the liquid formulation to gel.

In an alternate embodiment, the LPCP is poured on a collagen matrix, which activates the platelets in the LPCP, thereby forming a formulation which is applied directly to the wound or to a bandage or dressing for application to the wound. In this embodiment, as with those discussed above, the LPCP may first be mixed with a chemical platelet-activation agent, for example, thrombin or TRAP before application to the collagen matrix. Other additives, including collagen granules such as KOLLAGEN™ or calcium alginate such as SORBSAN®, with or without thrombin or TRAP, may be mixed with the LPCP to produce a formulation which is applied to the wound either directly or using an appropriate bandage or dressing. The formulation in each case is typically reapplied as often as is necessary to facilitate complete healing of the wound.

Preparation of Low Platelet Concentration Plasma

For each embodiment of the invention, a typical dosage formulation for the wound-healing formulation is prepared by initially drawing from a patient to be treated, a quantity of blood which depends on the number and size of the wound or wounds to be treated. Generally, the quantity of blood sufficient to obtain approximately 10 ml of platelet-plasma supernatant after centrifugation is about 20 ml. Ten (10) ml is the amount of wound healing formulation sufficient for the treatment of one wound of less than 10 cm in width or diameter. For larger or numerous wounds, the quantity of blood required will vary accordingly.

The method of preparation of the wound healing formulation of the present invention comprises obtaining a volume of blood from the patient whose wound is to be treated. The blood is first drawn into a container containing an anticoagulant such as sodium citrate or modified acid-citrate dextrose solution. The blood drawing or harvesting step is typically carried out using a butterfly needle and blue stopper-topped vacuum tubes, after which the harvested whole blood is centrifuged using parameters and centrifugation equipment well-known to those skilled in the art for platelet separation from the other blood components.

Typically, the harvested blood is centrifuged at about 135-280×g for about 20 seconds to 5 minutes at about 20-37° C. to obtain approximately 10 ml of low platelet concentration plasma (LPCP). The plasma and buffy-coat, that is, up to 70% of the liquid portion of the blood sample above the red blood cells, are then harvested. The LPCP is then deposited in a suitable container, and in a first embodiment of the invention, a formulation solution is next prepared from the LPCP by adding and mixing a quantity of thrombin, either bovine or autologous, the preparation of which is described below. CaCl₂ in an amount sufficient to enhance activation of the platelets in the formulation solution (about 0.5-1 ml of 10% CaCl₂) may also be a dded. Optionally, about 0.5 ml of a sterile injectable CKU-copper sodium chloride solution containing a triple copper peptide (Procyte Corp., Redmond, Wash.) is added at a concentration of from about 0.002% to about 2.00%, and preferably about 0.01% to 0.05%, to the formulation solution to enhance wound healing. In some situations, the addition of other additives to the formulation is desirable. These include antibiotics and recombinant growth factors (e.g., PDGF, EGF, VEGF, FGF, TGFβ).

Preparation of Autologous Thrombin

Autologous thrombin for use as a platelet activation agent may be prepared as follows. An amount of blood sufficient to yield 10 ml of platelet rich plasma (PRP) is drawn from the patient into a plastic syringe or non-clotting container. The blood is centrifuged in accordance with methods well known in the art for the preparation of PRP. 10 ml of PRP is removed and injected into a dry, non-additive, container to which is added 0.33 ml of 10% CaCl₂. The tube is agitated to ensure that the PRP and CaCl₂ solution are well mixed. Once the mixture has coagulated, the clot is squeezed out and discarded. What remains is autologous thrombin. To prepare the wound healing composition of the present invention, mix 1 ml of the autologous thrombin with 6-10 ml of LPCP.

While effective in the treatment of a variety of different situations, for example, in surgical settings, the application to incisional sites and to different types of wounds, the wound-healing formulation and method of the present invention is particularly effective in the treatment of chronic wounds. By definition, a chronic wound is one that shows no significant signs of healing after four weeks or has not healed completely after eight weeks. The three most common types of chronic wounds are diabetic foot ulcers, venous stasis ulcers and pressure sores, formerly known as decubitus ulcers.

The wound healing formulation and method of the present invention may also be used in the operating room during or after surgery, particularly in those patients whom the attending physician believes are at risk for wound problems or breakdown. Examples of these types of patients include surgical patients who suffer from malnutrition, steroid-dependent patients, patients with diminished vascular inflow, etc. Under these circumstances, the gel can be prepared at the time of surgery and applied around the repair sites of the bowel, vascular structures, wound closure sites, etc.

Another application for the wound healing formulation and method of the present invention includes orthopedic surgical procedures and bone grafting procedures. Insulin-like growth factor (ILGF) is well documented to stimulate osteoblastic activity. It has been found that use of autologous platelet gels in bone grafting procedures has resulted in healing of the bone graft in less than one half of the normal bone graft healing time, as compared to those procedures in which the gel is not used. In addition, the gel may be applied around the bone fragments of any fracture treated with open reduction and internal fixation of the fracture. In the event that internal hardware is used for the stabilization of bony structures, such as would occur in placing a metal rod into a femur or the insertion of a prosthesis used to stabilize the spine, etc., application of the gel around the hardware will promote faster stabilization due to enhanced osteoblastic activity and bony ingrowth around and into the hardware.

The wound-healing formulation of the present invention is particularly effective in the treatment of any type of chronic wound (decubitus wounds, diabetic foot ulcers, venous stasis ulcers, post-operative surgical wound dehiscence, brown recluse spider bites, etc.), which has shown minimal, or no signs of healing using the more standard or conventional types of wound therapy and management. Prior to preparation and application of the prepared formulation, the wound should be examined for infection. The decision about the status, degree or severity of infection and the degree to which the infection should be treated prior to treatment with the wound healing composition of the invention should be made either by a physician or his or her designated assistant (i.e., nurse practitioner).

At the time of the first and any subsequent treatments, the wound should first be debrided and all necrotic tissue should be completely removed down to healthy, bleeding tissue. The formulation, prepared as described above, is next applied directly into the wound area. Alternatively, the formulation may be used to saturate a gauze sponge or sponges, which are then placed in the wound, or sprayed on the wound or on the dressing that would be applied to the wound. The wound area is then covered with an occlusive dressing, which may remain in place for 2-7 days. When the dressing is changed the first time, the wound is cleansed with saline and the same or an alternate form of wound dressing may be used. The treatment may be repeated every 1-2 times per week over a total period of typically about 6-12 weeks as deemed necessary by the patient's physician for complete or nearly complete wound healing.

The invention will be further understood by reference to the following examples, which should not be construed to limit the invention.

### EXAMPLE 1

### Formulation Preparation Using Bovine Thrombin. for Treatment of Wounds

A diabetic patient presented with a chronic foot ulcer having a diameter of 4 cm. The foot ulcer was first debrided and all necrotic tissue was completely removed down to healthy, bleeding tissue. A wound healing formulation was prepared from the patient's blood. A quantity of blood sufficient to obtain approximately 10 ml of LPCP after centrifugation was drawn from the patient using a butterfly needle and blue stopper-topped vacuum tubes, after which the harvested blood was centrifuged at about 135×g for about 1 minute at about 20° C. to obtain the approximately 10 ml of LPCP. About 10 ml of a formulation solution was next prepared by mixing a quantity of thrombin and CaCl₂ in the concentration of about 1000 unit of thrombin per ml per 10 ml and 0.5 ml of CKU-copper. The formulation solution was then applied directly to the wound area and covered with an occlusive dressing, which remained in place for 4 days. At that time, the dressing was removed, the wound was cleansed with saline and another batch of liquid formulation was prepared according to the procedure described above. The liquid formulation was re-applied and covered with an occlusive dressing. The process was repeated every four days until complete healing took place over a total period of about 6 weeks.

### EXAMPLE 2

### Liquid Formulation Preparation Using TRAP for Treatment of Wounds

A wound healing liquid formulation is prepared from a patient afflicted with a chronic ulcer having a diameter of 4 cm. A quantity of blood sufficient to obtain approximately 10 ml of low platelet concentrate plasma after centrifugation was drawn from the patient using a butterfly needle and blue stopper-topped vacuum tubes, after which the harvested blood was centrifuged at about 135×g for about 1 minute at about 20° C. About 10 ml of a formulation solution was next prepared by mixing a quantity of thrombin receptor activating peptide (TRAP) in a concentration of about 0.4 mg per 10 ml of formulation solution. The liquid formulation solution was then applied to the wound, as described above.

### EXAMPLE 3

### Preparation Using Glass Beads

A wound healing gel formulation was prepared from a patient afflicted with a bedsore having a diameter of about 10 cm. A quantity of blood sufficient to obtain approximately 10 ml of platelet-plasma supernatant after centrifugation was drawn from the patient using a butterfly needle and blue stopper-topped vacuum tubes, after which the harvested blood was centrifuged at about 135×g for about 1 minute at about 20° C. to obtain the approximately 10 ml of low platelet concentration plasma (LPCP). The LPCP was then injected into an uncoated glass Petri dish through a syringe containing glass beads, and the glass beads and uncoated glass in the Petri dish activated the platelets in the low platelet concentration plasma and caused it to form a gel. The gel was applied to the wound according to the procedure in EXAMPLE 1 above.

### EXAMPLE 4

### Formulation Preparation Using TRAP and Glass Beads for Treatment of Wounds

A wound healing gel formulation was prepared from a patient afflicted with necrosis resulting from a Brown Recluse Spider bite. The wound area had a diameter of about 3 cm. A quantity of blood sufficient to obtain approximately 10 ml of low platelet concentration plasma after centrifugation was drawn from the patient using a butterfly needle and blue stopper-topped vacuum tubes, after which the harvested blood was centrifuged at about 135×g for about 1 minute at about 20° C. to obtain the approximately 10 ml of low platelet concnetration plasma. About 10 ml of a liquid formulation solution was next prepared by mixing a quantity of thrombin receptor activating peptide (TRAP) in a concentration of about 0.4 mg per 10 ml of platelet/plasma. The liquid formulation solution was then injected into an uncoated glass Petri dish through a syringe containing glass beads, which glass beads and uncoated glass in the Petri dish further enhanced activation of the platelets in the formulation solution and caused the liquid formulation solution to gel. The resulting formulation gel was applied to the wound as described in EXAMPLE 1 above.

### EXAMPLE 5

### Liquid Formulation Preparation Using Collagen Matrix

A wound healing gel formulation was prepared from a patient afflicted with necrosis resulting from a Brown Recluse Spider bite. The wound area had a diameter of about 5 cm. A quantity of blood sufficient to obtain approximately 10 ml of LPCP after centrifugation was drawn from the patient using a butterfly needle and blue stopper-topped vacuum tubes, after which the harvested blood was centrifuged at about 135×g for about 1 minute at about 20° C. to obtain approximately 10 ml of low platelet concentration plasma (LPCP). The LPCP preparation was then poured onto a collagen matrix, which activated the platelets in the LPCP and was applied to the wound using a bandage or dressing according to the procedure in EXAMPLE 1.

### EXAMPLE 6

### Liquid Formulation Preparation Using Collagen Matrix and TRAP

A wound healing formulation was prepared from a patient afflicted with a venous stasis ulcer having a diameter of about 6 cm. A quantity of blood sufficient to obtain approximately 10 ml of low platelet concentration plasma after centrifugation was drawn from the patient using a butterfly needle and blue stopper-topped vacuum tubes, after which the harvested blood was centrifuged at about 135×g for about 1 minute at about 20° C. to obtain approximately 10 ml of low platelet concentration plasma (LPCP). About 10 ml of a formulation solution was next prepared by mixing a quantity of thrombin receptor activating peptide (TRAP) in a concentration of about 0.4 mg per 10 ml of low platelet concentration plasma. The liquid formulation solution was then poured onto a collagen matrix, which enhanced activation of the platelets in the LPCP and was applied to the wound using a bandage or dressing according to the procedure in EXAMPLE 1.

### EXAMPLE 7

### Formulation Preparation Using Collagen Granules

A wound healing gel formulation is prepared from a patient afflicted with a chronic diabetic foot ulcer having a diameter of 4 cm. A quantity of blood sufficient to obtain approximately 10 ml of low platelet concentration plasma after centrifugation was drawn from the patient using a butterfly needle and blue stopper-topped vacuum tubes, after which the harvested blood was centrifuged at about 135×g for about 1 minute at about 20° C. to obtain the approximately 10 ml of low platelet concentration plasma. About 10 ml of a formulation solution was next prepared by mixing a quantity of KOLLAGEN® collagen granules with the PEP supernatant, wherein the collagen granules activated the platelets in low platelet concentration plasma preparation. The liquid formulation solution was then applied to the wound in the manner described with respect to EXAMPLE 1.

### EXAMPLE 8

### Gel Formulation Preparation Using TRAP and Sodium Chloride Solution for Treatment of Wounds

A wound healing gel formulation was prepared from a patient afflicted with a chronic diabetic foot ulcer having a diameter of about 7 cm. A quantity of blood sufficient to obtain approximately 10 ml of low platelet concentration plasma after centrifugation was drawn from the patient using a butterfly needle and blue stopper-topped vacuum tubes, after which the harvested blood was centrifuged at about 135×g for about 1 minute at about 20° C. to obtain the approximately 10 ml of low platelet concentration plasma. About 10 ml of a formulation solution was next prepared by mixing a quantity of thrombin receptor activating peptide (TRAP) in a concentration of about 0.4 mg per 10 ml of low platelet concentration plasma, wherein TRAP activated platelets in the low platelet concentration plasma. A sterile injectable CKU-copper sodium chloride solution containing a triple copper peptide (e.g., glycyl-histidyl-lysine-Cu, Procyte Corp. Redland, Wash.), zinc and silver in a concentration of about 0.02% in a volume of typically about 1 ml was added to the formulation solution to enhance wound healing. The liquid formulation solution was then poured into an uncoated glass Petri dish and solidified in about two seconds to form a gel, which was applied to the wound as previously described.

1. A method of producing a wound healing composition comprising:
a) obtaining whole blood from a patient;
b) obtaining a low platelet concentration plasma preparation from said blood; and
c) activating said low platelet concentration plasma preparation.

2. A topical wound healing composition comprising:
a) a low platelet concentration plasma preparation; and
b) a platelet activating agent.

3. The composition of claim 2 further comprising CaCl₂ in an amount sufficient to enhance activation of said platelets.

4. The composition of claim 2 further comprising glycyl-histidyl-lysine-Cu.

5. The composition of claim 2 further comprising an antibiotic.

6. The composition of claim 2 further comprising a recombinant growth factor.

7. The composition of claim 2 further comprising collagen granules.

8. The composition of claim 2 wherein said platelet activating agent is autologous thrombin.

9. The composition of claim 2 wherein said platelet activating agent is bovine thrombin.

10. The composition of claim 2 wherein said platelet activating agent is a thrombin receptor activating peptide.

11. A method for treating a wound of a patient which comprises applying to the wound an effective amount of a wound healing composition comprising a low platelet concentration plasma preparation.

12. A method for treating a wound of a patient which comprises:
a) obtaining whole blood from the patient;
b) obtaining a low platelet concentration plasma preparation from said blood;
c) activating said low platelet concentration plasma preparation to form a wound healing composition; and
d) applying said wound healing composition to the wound of the patient.

13. The method of claim 12 wherein said wound healing composition further comprises glycyl-histidyl-lysine-Cu.
